(19) United States
(12) Patent Application Publication
(10) Pub. No.: US 2024/0410052 A1
(43) Pub. Date: Dec. 12, 2024

(54) MULTIFUNCTIONAL COATING, METHOD OF MANUFACTURING THEREOF, RELATED COATED ITEMS AND USES

(71) Applicant: PICOSUN OY, Espoo (FI)

(72) Inventors: Jesse KALLIOMÄKI, Helsinki (FI); Riina RITASALO, Espoo (FI); Tom BLOMBERG, Vantaa (FI); Ilkka MANNINEN, Espoo (FI); Juhani TASKINEN, Espoo (FI)

(21) Appl. No.: 18/697,992

(22) PCT Filed: Oct. 28, 2022
(86) PCT No.: PCT/EP2022/080209

§ 371 Date: Apr. 2, 2024

(30) Foreign Application Priority Data
Oct. 29, 2021 (FI) 20216125

(51) Int. Cl. C23C 16/455; A61B 8/00; C23C 16/02; C23C 16/40

(52) U.S. Cl. CPC C23C 16/45529; A61B 8/4483; C23C 16/0272; C23C 16/402; C23C 16/403; C23C 16/405; C23C 16/45555

(57) ABSTRACT

A method for forming a coating on a substrate is provided, the method comprises: using a molecular layer deposition (MLD) process depositing at least one layer directly or indirectly on a surface, and an atomic layer deposition (ALD) process, depositing an inorganic film on/over the at least one layer. In the formed coating conditions are established which allow unbound, unreacted and/or partially reacted precursors to enter chemical interaction with harmful environmental species penetrated into the coating at defective sites thereof and seal said defective sites through formation of a sealing compound. A laminate coating, uses thereof and coated items are further provided.

### FIELD OF THE INVENTION

The present invention generally relates to manufacturing of coated items by chemical deposition methods. In particular, the invention concerns deposition of multilayer laminate coatings onto sensitive and potentially flexible substrates. These multilayer laminate coatings render a protective layer. This protective layer confers a multitude of functions including as a corrosion or a moisture barrier for a range of sensitive and flexible substrates, and further the protective barrier layer confers a capability to of sustain a reasonable amount of damage.

### BACKGROUND OF THE INVENTION

Generation of protective coatings on a variety of substrates through the methods of chemical deposition methods in vapour phase, such as Atomic Layer Deposition (ALD) and Molecular Layer Deposition (MLD), are extensively described in the art.

Atomic layer deposition technology is based on alternating, self-saturative surface reactions, wherein different precursors (reactants) provided as molecular compounds or elements in a nonreactive (inert) gaseous carrier are sequentially pulsed into a reaction space accommodating a substrate. Deposition of a precursor is followed by purging the substrate by inert gas. Conventional ALD cycle (a deposition cycle) proceeds in two half-reactions (pulse first precursor—purge; pulse second precursor—purge), whereby an atomic monolayer of material is formed in a self-limiting (self-saturating) manner, typically being 0.05-0.2 nm thick. The cycle is repeated as many times as required for obtaining a film with a predetermined thickness. Typical substrate exposure time for each precursor ranges within 0.01-10 seconds. ALD technique has been developed for growing inorganic materials at the atomic level, with the most common precursors including metal oxides, elemental metals, metal nitrides and metal sulfides. ALD-deposited films are fully conformal and pinhole-free.

Molecular layer deposition is also a sequential self-limiting vapour-phase deposition method allowing for production of (ultra-)thin organic and hybrid organic-inorganic films. Similar to ALD, the layer-by-layer nature of molecular layer deposition method allows production of highly conformal thin films with sub-nanometer thickness control. While in production of pure organic (polymeric) structures, a combination of two or more organic precursors is adopted, hybrid organic-inorganic films are typically synthesized using a combination of inorganic compounds with organic polymers. Common organic precursors are polymer molecules that contain —OH, —COOH, CONH₂, —CHO, —NH₂, —SH, —CN functional groups, for example.

Different packaging applications, in particular those utilized in medical packaging (containers, pharmaceutical packaging, etc.) need sterile packaging solutions. In many cases the coating solution needs to be transparent, thin and scalable, resistant to corrosive liquids and ambient moisture, non-cytotoxic, and it is crucial to be able to coat the different shaped surfaces on conformal and controlled manner.

US 2010/178481 A1 (George et al.) describes ALD-MLD coatings applied on flexible substrates. The coating comprises multiple layers of inorganic material other than silica, such as metal oxide, a metal or a semi-metal nitride, wherein at least some of the adjacent layers are separated from each other by at least one silica layer and at least one layer of an organic polymer. The inorganic and silica layers are deposited by ALD, the organic polymer or a hybrid inorganic-organic polymer layer is deposited by MLD.

US 2013/296988 A1 (Weber et al) describes a medical implant comprising a nanolaminate with at least one ceramic layer deposited by ALD and at least one polymer layer deposited by either MLD, sol gel processes, liquid-source misted chemical deposition (LSMCD) or plasma-enhanced chemical vapour deposition (PECVD). Nanoclay and/or nanodiamond is incorporated into the polymer material in addition to or in place of the ceramic material to improve the strength and wear resistance.

US 2013/333835 A1 (Carcia et al.) describes a process for manufacturing either rigid or flexible protective barrier coatings comprising a hybrid inorganic-organic, polymeric alloys, by combining ALD and MLD techniques. The transparent alloy may be formed directly on the object to be protected or on a carrier substrate that may subsequently employed to protect an object. A barrier capping structure is similar to the actual barrier structure.

Nevertheless, although previously recognized multilayer coatings deposited using the above described atomic- and molecular layer deposition methods have proved to act as efficient corrosion resistant barriers, in many instances these coatings become deprived of their protective ability in case of mechanical damage of the coating.

In this regard, an update in the field of fabricating protective encapsulation is still desired, in particular, in view of addressing challenges associated with the application of said coatings in manufacturing of biomedical devices, such as implantable bioelectronics solutions.

### SUMMARY OF THE INVENTION

An objective of the present invention is to solve or to at least alleviate each of the problems arising from the limitations and disadvantages of the related art. The objective is achieved by means of a method for forming a coating on a substrate, a self-healing laminate coating, related items and uses in accordance with the respective independent claims appended to the present description.

In an aspect of the invention, a method for forming a coating on a substrate using a molecular layer deposition process and atomic layer deposition, according to what is defined in independent claim 1.

In an embodiment, a method for forming a coating on a substrate, comprises:
(i) using a molecular layer deposition (MLD) process, depositing at least one layer composed of essentially porous bulk of material directly or indirectly on a surface of the substrate, and
(ii) using an atomic layer deposition (ALD) process, depositing an inorganic film on/over the at least one layer formed in step (i),
whereby a coating is formed, wherein, in said at least one layer composed of essentially porous bulk of material, the unbound, unreacted and/or partially reacted precursors enter chemical interaction with harmful environmental species penetrated into the coating at defective sites thereof and seal said defective sites through formation of a sealing compound.

Said harmful environmental species may comprise any one of: water molecules, hydroxyl radicals, nitrous oxide species, biological molecules, such as for example proteins, and the like, originated from an environment surrounding the substrate(s) coated with the laminate coating and penetrated into the coating at the defective sites.

In some instances, in said coating, conditions are established that allow infiltration of the unbound, unreacted and/or partially reacted precursors into the essentially porous bulk of material during step (i).

In embodiment, the inorganic film formed in step (ii) comprises at least one deposition layer.

In embodiment, the inorganic film formed in step (ii) comprises a plurality of deposition layers arranged into a stack, wherein each deposition layer in the stack has the same or different composition.

In embodiment, the deposition layer or layers forming the inorganic film in step (ii) are composed of any compound selected from the group consisting of: aluminium (III) oxide (Al₂O₃), titanium(IV) oxide (TiO₂), hafnium (IV) oxide (HfO₂), tantalum (V) oxide (Ta₂O₅), zirconium (IV) oxide (ZrO₂), and silicon dioxide (SiO₂).

In embodiment, the plurality of deposition layers forming the inorganic film in step (ii) includes the deposition layers composed of aluminium (III) oxide (Al₂O₃) alternating with the deposition layers composed of a metal oxide compound different from aluminium (III) oxide.

In embodiment, the plurality of deposition layers forming the inorganic film in step (ii) includes the deposition layers composed of aluminium (III) oxide (Al₂O₃) alternating with the deposition layers composed of any one of titanium(IV) oxide (TiO₂), hafnium (IV) oxide (HfO₂), tantalum (V) oxide (Ta₂O₅), and zirconium (IV) oxide (ZrO₂).

In embodiment, the plurality of deposition layers forming the inorganic film in step (ii) includes the deposition layers composed of aluminium (III) oxide (Al₂O₃) alternating with the deposition layers of hafnium (IV) oxide (HfO₂).

In embodiment, method comprises repeating steps (i) and (ii) until the coating having a desired total thickness has been formed. In embodiment, step (ii) is performed before step (i). In embodiments, the MLD layer deposited at step (i) alternates with every two or three ALD deposition layers deposited at step (ii).

In embodiment, the method further comprises pretreatment of the substrate. In embodiments, pretreatment of the substrate comprises treating the substrate with any one of ozone (O₃), oxygen (O₂), and/or by depositing, optionally using the atomic layer deposition process, onto its surface a primer layer that enhances adhesion of the essentially porous material layer formed in step (i) to the surface of the substrate.

In embodiment, the method further comprises depositing a polymer film on/over the coating as a topmost layer. In embodiment, the polymer film constituting the topmost layer consists of polydimethylsiloxane (PDMS) or polyurethane (PU).

In another aspect, a self-healing laminate coating is provided, according to what is defined in independent claim 14.

In embodiment, the self-healing laminate coating is formed on a substrate and it comprises: (a) at least one layer composed of essentially porous bulk of material deposited using a molecular layer deposition (MLD) process, and (b) an inorganic film deposited using an atomic layer deposition (ALD) process, wherein, in said at least one layer composed of essentially porous bulk of material, unbound, unreacted and/or partially reacted precursors enter chemical interaction with harmful environmental species penetrated into the coating at defective sites thereof and seal said defective sites through formation of a sealing compound.

In embodiment, in the laminate coating, the inorganic film (b) comprises at least one deposition layer.

In embodiment, in the laminate coating, the inorganic film (b) comprises a plurality of deposition layers arranged into a stack, wherein each deposition layer in the stack has same or different composition.

In embodiment, in the laminate coating, the deposition layer or layers forming the inorganic film (b) are composed of any compound selected from the group consisting of: aluminium (III) oxide (Al₂O₃), titanium(IV) oxide (TiO₂), hafnium (IV) oxide (HfO₂), tantalum (V) oxide (Ta₂O₅), zirconium (IV) oxide (ZrO₂), and silicon dioxide (SiO₂).

In embodiment, in the laminate coating, the inorganic film (b) comprises the deposition layers composed of aluminium (III) oxide (Al₂O₃) alternating with the deposition layers composed of a metal oxide compound different from aluminium (III) oxide.

In embodiment, in the laminate coating, the inorganic film (b) comprises the deposition layers composed of aluminium (III) oxide (Al₂O₃) alternating with the deposition layers composed of any one of titanium(IV) oxide (TiO₂), hafnium (IV) oxide (HfO₂), tantalum (V) oxide (Ta₂O₅), and zirconium (IV) oxide (ZrO₂).

In embodiment, in the laminate coating, the inorganic film (b) comprises the deposition layers composed of aluminium (III) oxide (Al₂O₃) alternating with the deposition layers of hafnium (IV) oxide (HfO₂).

In embodiment, in the laminate coating, the inorganic film (b) comprises deposition layers composed of aluminium (III) oxide (Al₂O₃) alternating with the deposition layers of tantalum (V) oxide (Ta₂O₅).

In embodiment, the laminate coating comprises the inorganic film (b) deposited on/over the substrate and/or on/over the at least one layer of essentially porous material (a).

In embodiment, the laminate coating comprises the inorganic film (b) deposited using the atomic layer deposition (ALD) process on/over the substrate. In embodiments, said inorganic film (b) is formed with a plurality of deposition layers arranged into at least one stack having any one of the following compositions: Al₂O₃—SiO₂—Al₂O₃—TiO₂; Al₂O₃—HfO₂—Al₂O₃—ZrO₂; Al₂O₃—HfO₂—ZrO₂; or TiO₂+[(Al₂O₃—TiO₂)]. In embodiments, the above-mentioned sequences of deposition layers in said inorganic film (b) are arranged into repeating stacks.

In embodiments, the laminate coating comprising the inorganic film (b) deposited using the ALD process on/over the substrate further comprises at least one layer (a) composed of essentially porous bulk of material deposited using a molecular layer deposition (MLD) process.

In embodiments, the laminate coating comprises the at least one layer (a) composed of essentially porous bulk of material deposited directly on the substrate, or on top of the inorganic film (b), optionally, on top of a first stack of the plurality of deposition layers provided within said inorganic film (b).

In embodiment, the laminate coating further comprises a primer layer formed on the substrate surface to enhance adhesion of the essentially porous material layer (a) to the surface of the substrate, said primer layer being optionally deposition using the atomic layer deposition process.

In embodiment, the laminate coating further comprises a polymer film deposited on/over the coating as a topmost layer. The polymer film constituting the topmost layer may consist of polydimethylsiloxane (PDMS).

In embodiments, in said laminate coating, the substrate is selected from the group consisting of: a medical device, a medical packaging, an Organic Light Emitting Diode (OLED), and a sensor.

In embodiment, the sealing compound formed in the essentially porous bulk of material (layer (a)) is a product of chemical interaction between the unbound, unreacted and not fully (partially) reacted precursors and harmful environmental species, wherein said harmful environmental species comprise any one of: water molecules, hydroxyl radicals, nitrous oxide species and the like, originated from an environment surrounding the substrate(s) coated with the laminate coating and penetrated into the coating at the defective sites.

In a further aspect, use of the laminate coating according to some previously defined aspect and embodiments as desiccant is provided. In still further aspect, use of the laminate coating according to some previously defined aspect and embodiments is provided in packaging, particularly, in medical packaging. In still further aspects, use of the laminate coating according to some previously defined aspect and embodiments is provided in applications for any one of: catalyst support; solid electrolyte for microbatteries; lithium-ion battery separators; free-standing films; water repellent layer on sensors; fluorescent thin films; barrier film for flexible electronics; barrier for optics, in particular, LEDs, Quantum Dots, and/or nanorod-LEDs; barrier for nanoparticles, such as phosphor nanoparticles; flexible smart windows; smart contact lenses; smart sensors, and space related applications.

In some other aspect, a packaging item is having its surface coated with the laminate coating according to some previously defined aspect and embodiments, and/or with the laminate coating formed by the method according to some previously defined aspect and embodiments. In embodiment, the packaging item is selected from the group consisting of: a container, optionally a pumped container, a tray, optionally, a multi-compartmental tray, an ampule, a vial, a syringe, a blister pack, an individually wrapped pack, and a pouch. In embodiments, packaging item has a substrate comprising or consisting of glass or polymer, such as polyurethane (PU), liquid crystal polymer (LCP), and/or polyethylene terephthalate (PET).

In some other aspect, a medical device, such as an implantable medical device, is provided, said medical device having its surface coated with the laminate coating according to some previously defined aspect and embodiments, and/or with the laminate coating formed by the method according to some previously defined aspect and embodiments. In embodiment, the medical device is configured as an ultrasonic medical device, optionally, as a piezoelectric micromachined ultrasonic transducer (PMUT) or a capacitive micromachined ultrasonic transducer (CMUT).

The utility of the present invention arises from a variety of reasons depending on each particular embodiment thereof.

The primary advantages of ALD are all derived from the sequential, self-saturating, gas-surface reaction control of the deposition process. This growth mode of the film enables extremely conformal and uniform films to be coated on three-dimensional objects having any shape, therefore, the ALD technology has a high potential in manufacturing of high-quality coatings required for various applications, in particular, medical applications. Due to relatively low temperatures utilized during ALD depositions (85-150° C.), the coatings can be conformally deposited also on sensitive substrate materials. Additionally, since the ALD coatings are generally amorphous (not crystalline), these provide no easy pathway for harmful, e.g. corrosive species, therethrough.

Overall, the invention offers a method for producing (nano)laminate coatings with enhanced resistance to (micro)defects and also acting as reliable low temperature barriers against harmful environmental species, such as moisture and/or ions, UV light, heat, oxygen, and the like. The invention further offers a deposition method for producing a self-repairing/self-healing coating film compatible with the methods of low-temperature thermal processing.

The method presented hereby concerns fabrication of a thin film encapsulation coating, particularly on sensitive and optionally flexible substrates, which coating is capable of self-sustaining and self-recovering from a reasonable amount of damage.

Through thorough selection of coating materials, it is possible to ALD-deposit a plurality of extremely thin inorganic layers, which allow the coating structure to remain flexible, while the periodic structure increases the volume of impermeable inorganic material (e.g. metal oxide material) to ensure good barrier properties. On the other hand, the MLD layers act as “decoupling” layers between the ALD-deposited inorganic barrier films to obstruct defect propagation through the coating and to increase a Water vapor transmission rate (WVTR) lag-time or moisture permeation by creating a tortuous path.

The (nano)laminate coating is particularly suitable for use in medical segment, such as for implantable electronics in cardiology and neurology, to serve as a protective, non-toxic and long-lasting medical coating solution.

Improved reliability in operation and increased lifetimes are of particular importance for implantable medical devices, since replacement of an implant is often associated with surgery; therefore, improving reliability of such devices along with performance characteristics is highly beneficial both for patients and for the healthcare systems.

The invention further provides for increased lifespan and enhanced performance of moisture sensitive components used in industrial- and consumer electronics.

In the present disclosure, materials with a layer thickness below 1 micrometer (μm) are referred to as “thin films”.

The expressions “reactive fluid” and “precursor fluids” are indicative in the present disclosure of a fluidic flow comprising at least one chemical compound (a precursor compound), hereafter, a precursor, in an inert carrier.

In the present disclosure, the term “biocompatibility” is used in its common meaning, viz. defined as an ability of a device or a system of technical nature exposed to biological environment to perform its function without major and clinically adverse manifestations both short and long term. Assessing biocompatibility largely involves analyzing the ability of materials constituting the device/system to interact with biological environment without developing unwanted inflammation or complications in the body.

Unless otherwise explicitly indicated, in the present disclosure, the term “environment” refers to the environment surrounding the substrate coated with a coating, according to the embodiments.

In the present disclosure, the term “body” (in the context of the expressions “body implantable”, “body environment”, etc.) is used primarily with regard to a human.

However, the concept of the present invention is fully applicable to the items, such as medical devices, designed and/or used with a nonhuman mammal or other animal.

The expression “a number of” refers herein to any positive integer starting from one (1), e.g. to one, two, or three; whereas the expression “a plurality of” refers herein to any positive integer starting from two (2), e.g. to two, three, four, and so on.

The terms “first” and “second” are not intended to denote any order, quantity, or importance, but rather are used to merely distinguish one element from another, unless explicitly stated otherwise.

### BRIEF DESCRIPTION OF THE DRAWINGS

FIGS. 1A-1C schematically illustrate, at 10, a concept of a self-healing laminate coating 10, according to the embodiments, on a substrate 20.

FIG. 2 schematically illustrate, at 10′, an ALD-nanolaminate coating on the substrate 20.

FIGS. 3A and 3B demonstrate experimental results for Water vapor transmission rate (WVTR) and defective area measurements obtained for the laminate coating according to the embodiments, in comparison with monolithic ALD structures.

### DETAILED DESCRIPTION OF THE EMBODIMENTS

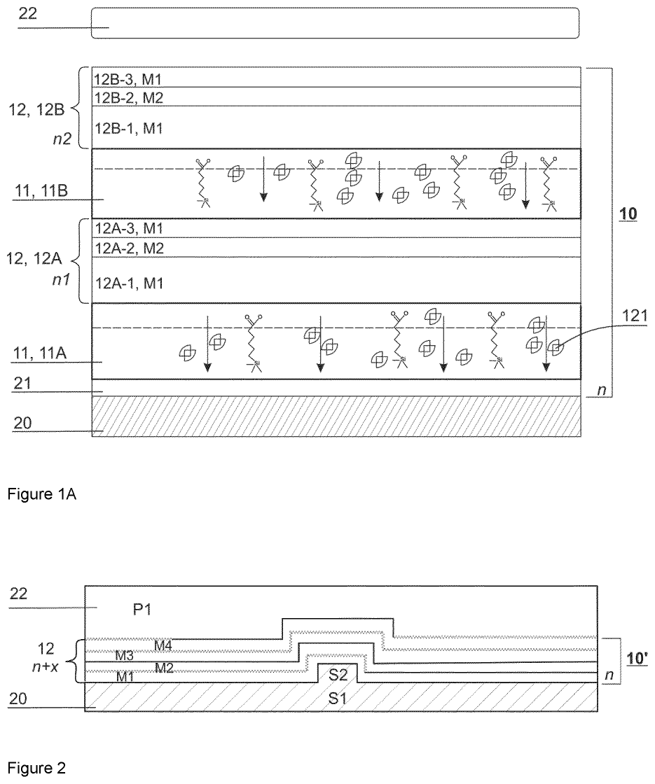

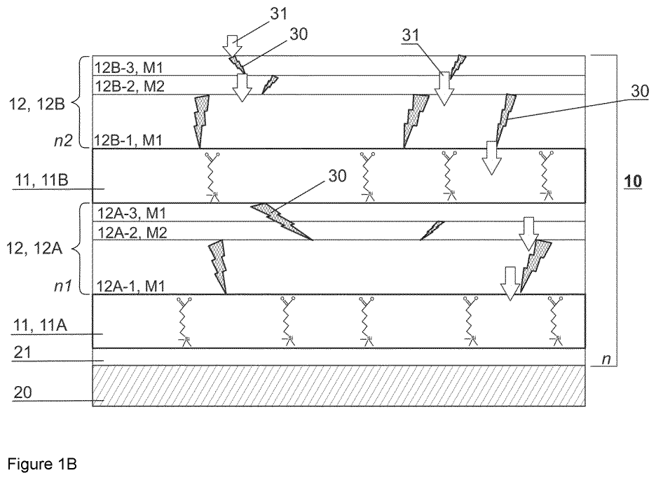

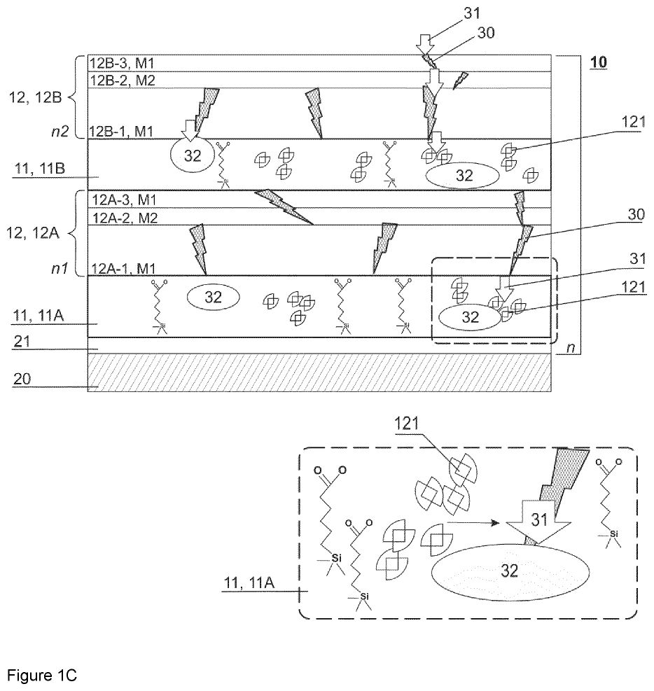

FIGS. 1A, 1B and 1C illustrate, at 10, a concept underlying a protective laminate coating hereafter, a coating, formed on a substrate 20 in accordance with embodiments. In some instances, the protective coating is provided with a self-healing functionality.

The coating 10 is advantageously designed for optionally flexible components (substrates) susceptible to erosion or corrosion induced by a variety of harmful environmental species originating from an environment surrounding the substrate(s) 20 coated with the coating 10. The environment can be e.g. the atmosphere, any gaseous medium, such as ambient air, for example, and/or any moisture-containing environment, such as an essentially liquid medium (essentially aqueous media, such as water, ion-containing media, saline media, etc.) or a solid medium (e.g. powders or particulates). For the sake of clarity, we note that also the atmosphere/the ambient surrounding the coated substrate can represent the moisture-containing medium. In some instances, the environment includes a variety of in vitro cultures (e.g. cell cultures) and in vivo media. The latter can be represented with body fluids and tissue.

The term “moisture” is used hereby to indicate the presence of liquid, particularly water in the environment surrounding the coated substrate. In some instances, the term pertains to an amount of water vapour present in the atmosphere.

The coating 10 is formed on the substrate (20) surface using the techniques of molecular layer deposition (MLD) and atomic layer deposition (ALD). By virtue of a thorough selection of precursors (reactants) for these deposition processes, the coating 10 can be rendered with a self-healing functionality. The coating 10 can be configured to coat a part of the substrate or the entire substrate.

The coating 10 comprises at least one layer or film 11 composed of an essentially porous bulk of material. The layer 11 is also referred to as an “essentially porous layer” or “an “MLD layer”. The layer 11 is deposited using the MLD process directly or indirectly on a surface of the substrate 20. Direct deposition refers to depositing the essentially porous layer 11 on/over the surface of the substrate 20; whereas indirect deposition is indicative of deposition of the same on/over a surface of a primer layer formed (directly) on the substrate 20, as described further below.

In some configurations, the MLD layer 11 is formed as a purely organic polymer film. In some other configurations, the MLD layer 11 is formed as a hybrid organic-inorganic film layer.

Deposition setup used for MLD and ALD reactions was the one based on an ALD installation described in the U.S. Pat. No. 8,211,235 (Lindfors), for example, or on the installation trademarked as Picosun R-200 Advanced ALD system available from Picosun Oy, Finland. The same setup allows for depositing materials at the atomic. and at the molecular level.

An exemplary deposition reactor comprises the reaction chamber that establishes the reaction space (deposition space), in which the production of the coating 10 described herewith takes place. The reactor further comprises a number of appliances configured to mediate fluidic flow (the flow of inert fluid(s) and reactive fluid(s), the latter containing precursor compounds) into/through the reaction chamber. These appliances are provided as a number of intake lines/feedline and associated switching and/or regulating devices, such as valves, for example. Unreacted/unbound precursors and generated by-products are typically withdrawn from the reaction chamber by means of a vacuum pump.

In a typical MLD cycle, the molecules of a first precursor react with the reactive (linking) sites on the substrate surface, whereby a molecular layer of the first precursor is generated on the substrate surface. After a purge, the molecules of a second precursor react with new reactive sites generated through deposition of the first precursor, thus creating a molecular layer of the second precursor. At the same time, the surface chemistry is recovered to the initial reactive groups. The cycle is completed with a second purge. By repeating these four (4) steps (MLD precursor 1—purge—MLD precursor 2—purge), a polymeric film can be grown at a molecular level.

To enable fabrication of hybrid organic-inorganic films, the MLD process utilizes polymeric precursors (e.g. metal alkoxide materials) in combination with some common precursors typically used in ALD (e.g. tetramethylammonium, TMA).

An exemplary MLD process for depositing the essentially porous film 11 as a hybrid organic-inorganic film layer is described hereinbelow.

The exemplary MLD process utilizes 7-octenyltrichlorosilane (7-OTS) as a polymer precursor. During a deposition cycle, first 7-OTS and water used a catalyst were introduced into a reaction space of a deposition reactor followed with ozone (O₃) treatment. 7-OTS has terminal vinyl (—CH═CH2) groups that can be converted into carboxylic acid groups (—COOH) using ozone. Deposition cycle continued by introducing into a reaction space a metal precursor followed with water. In the described process, trimethylaluminum (TMA) was used as metal precursor, since it reacts with carboxylic groups forming a linker that can bind the polymeric layers together. By repeating the TMA-H₂O sequences 1-4 times, for example, a monolayer of aluminium (III) oxide (Al₂O₃) was formed with abundance of reactive Al—OH sites. Subsequent deposition of the 7-OTS and water into the reaction space results in construction of a hybrid organic-inorganic MLD film (11), where the Al₂O₃ layer is embedded between the organic polymer layers. MLD deposition temperature is within a range of about 50° C. to about 200° C., primarily, about 90° C.

In similar manner, a hybrid poly(aluminum ethylene glycol) polymer can be grown through sequential exposures of the substrate to TMA and ethylene glycol (EG). MLD deposition temperatures for hybrid poly(aluminum ethylene glycol) polymers is within a range of about 85° C. to 175° C.

Other than TMA metal precursors used to deposit the essentially porous films 11 include, but are not limited to: titanium tetraisopropoxide (TTIP, Ti(OCH(CH₃)₂)₄), zirconium butoxide (Zr(C₄H₉O)₄), and diethyl zinc (DEZ, ZnEt₂), for example.

The coating 10 further comprises an inorganic film 12 deposited using the atomic layer deposition process on/over said at least one layer of essentially porous material 11. Inorganic film 12 is also referred to, in general terms, as an “ALD film/ALD layer”.

As mentioned herein above, ALD is a special chemical deposition method based on the sequential introduction of at least two reactive precursor species to at least one substrate. In some particular methods, such as in photon-enhanced ALD or plasma-enhanced ALD, one of these reactive precursors can be substituted by energy, leading to single precursor ALD processes. For example, deposition of a pure element, such as metal, requires only one precursor. Binary compounds, such as oxides can be created with one precursor chemical when the precursor chemical contains both of the elements of the binary material to be deposited. Thin films grown by ALD are dense, pinhole free and tend to have uniform thickness.

In ALD, the at least one substrate is typically exposed to temporally separated precursor pulses in a reaction vessel to deposit material on the substrate surfaces by sequential self-saturating surface reactions. In the context of present disclosure, the term ALD comprises all applicable atomic layer deposition based techniques and any equivalent or closely related technologies, such as, for example the following ALD sub-types: plasma-assisted ALD, PEALD (Plasma Enhanced Atomic Layer Deposition) and photon-enhanced Atomic Layer Deposition (known also as photo-ALD or flash enhanced ALD).

A basic ALD deposition cycle consists of four sequential steps, comprising directing the first- and second precursors into the reaction chamber one after another. The precursors enter the reaction chamber as preferably a gaseous substance comprising a predetermined precursor chemical carried by an inert carrier (gas). The chamber is purged with inert gas between precursor pulses. Delivery of the precursor chemicals into the reaction space and film growth on the substrate is/are regulated by means of regulating appliances, such as e.g. three-way ALD valves, mass-flow controllers or any other device suitable for this purpose. One deposition cycle (ALD precursor 1—purge—ALD precursor 2—purge) typically yields an atomic (mono)layer of inorganic material formed by half-reactions between the first- and second precursors.

The above described deposition cycle can be repeated until the deposition sequence has produced a thin inorganic film, such as film 12, of desired thickness. Deposition cycles can be as simple as described above or more complex. For example, the cycles can include three or more reactant vapour pulses separated by purging steps, or certain purge steps can be omitted. On the other hand, photo-enhanced ALD has a variety of options, such as only one active precursor, with various options for purging. All these deposition cycles form a timed deposition sequence that is controlled by a logic unit or a microprocessor.

The ALD process utilized to produce the coating employs deposition temperatures within a range of about 80-250° C. Deposition within a range of 85-125° C. enables or facilitates coating of heat-sensitive substrates, such as those containing polymers and/or electric circuits.

FIGS. 1A-1C illustrate the exemplary coating 10 comprising two MLD layers 11 (11A, 11B) alternating with a corresponding number (hereby, two) of the ALD layers 12 (12A, 12B). Laminate structures for the coating 10 can be conceived comprising 1-5 MLD layers alternating with a corresponding number of ALD layers. Existing deposition techniques allow for constructing the laminate coating structures including more than five (5) MLD layers 11 (interfaced with ALD films 12); however, the coatings comprising 1-5 MLD layers 11 alternating with the same amount of ALD films 12 provide sufficient protection from corrosion induced by common environmental agents, such as moisture, for example.

In some configurations, the coating 10 may be formed as a periodic layered structure, in where the essentially porous layers 11, 11A, 11B (MLD) are interfaced with essentially impervious (unless damaged and/or defective) conformal layers 12, 12A, 12B (ALD).

In embodiments, the inorganic film 12 deposited using the atomic layer deposition technique comprises at least one deposition layer.

Hence, the coating 10 can be implemented with the (ALD) film comprising one deposition layer (not shown). In such an event, the inorganic film 12 is formed with a single ALD deposition layer composed of a predetermined material, such as a metal oxide, for example. The inorganic film 12 can also be formed in a number of sequential deposition cycles, in which event the film 12 is constructed from several monolayers of the same material.

In some configurations, the inorganic film 12 comprises a plurality of deposition layers arranged into at least one stack. FIGS. 1A-1C show the coating 10, in which each of the ALD films 12 (12-A, 12B) is composed of three deposition layers 12A-1, 12A-2 and 12A-3 (for the film 12A) and 12B-1, 12B-2 and 12B-3 (for the film 12B) arranged atop each other to from a stack. The deposition layers 12-1, 12-2, 12-3 forming the inorganic film 12 (“ALD layer”) can have same or different composition.

To avoid repetition, we note that the reference numerals 12-1, 12-2, 12-3 pertain to designate individual deposition layers within the film 12A (to be understood as 12A-1, 12A-2 and 12A-3) and within the film 12B (to be understood as 12B-1, 12B-2 and 12B-2).

Each individual deposition layer, such as 12-1, 12-2, 12-3 (FIGS. 1A-1C) can be formed as a monolayer in one (ALD) deposition cycle (with a sequence of ALD precursor—purge—ALD precursor 2—purge). In some configurations, each deposition layer can be formed in a number of sequential deposition cycles produce a deposition layer (e.g. 12-1) of desired thickness (whereby the deposition layer is constructed from several monolayers).

In coating configurations shown on FIGS. 1A-1C, the inorganic film 12 itself represents a layered (laminate) structure, wherein the deposition layers composed of a material having a first composition (M1 standing for “Material 1”) may alternate with deposition layers composed of a material having a second composition (M2 standing for “Material 2”). In presented configurations, the deposition layers 12-1 and 12-3 are made of a first material (e.g. aluminium (III) oxide or alumina, Al₂O₃), while the deposition layer 12-2 is made of a second material (e.g. silicon (IV) dioxide or silica, SiO₂).

In some embodiments, the deposition layers forming the film 12 have different composition. Alternatively, coating configurations comprising all deposition layers (such as 12-1, 12-2, 12-3) composed of the same material may be conceived.

For the sake of clarity, we note that a total number (n1, n2) of the plurality of the ALD deposition layers, such as 12-1, 12-2, 12-3, may vary dependent on layer composition, a substrate to be coated and an application field of the latter. By way of example, total number (n1, n2) of deposition layers may vary within a range of 1-100. In most instances, n1, n2 varies within a range of 1-20. Thickness of each deposition layer 12-1, 12-2, 12-3 may vary within a range of 1-20 nm, typically, these layers are 1-5 nm thick.

In some exemplary configurations, the coating 10 comprises a number of MLD-deposited organic or hybrid (organic-inorganic) 1-20 nm films 11 (11A, 11B) alternating with inorganic ALD films 12 (12A, 12B) having thickness as described above. Within the coating 10, the inorganic films 12 may have uniform composition (each formed of a single metal oxide, for example). Additionally or alternatively, the film(s) 12 can be formed with a (nano)laminate stack comprised of 1-4 deposition layers (1-20 nm for each deposition layer).

For a skilled person it is clear that dependent on reaction conditions one may deposit a plurality of 1-3 nm deposition layers, for example, thus regulating the thickness of a resulting stack and hence—the resulting inorganic film 12.

To form the coating 10, the processes of molecular layer deposition and atomic layer deposition are repeated until a laminate coating structure having a desired total thickness (n) has been formed. Total thickness of the coating 10 can be within a range of about 10 nm to about 1000 nm.

To form the ALD films 12, the deposition layers can be produced from a selection of (metal) oxide species including, but not limited to: aluminium (III) oxide (Al₂O₃), titanium(IV) oxide (TiO₂), hafnium (IV) oxide (HfO₂), tantalum (V) oxide (Ta₂O₅), zirconium (IV) oxide (ZrO₂), niobium (V) oxide (Nb₂O₅), and silicon dioxide (SiO₂). Metal nitride compounds, such as titanium nitride and aluminium nitride, for example, can also be utilized. Utilization of any other appropriate compound is not excluded.

In embodiments, the coating 10 can be provided wherein the plurality of deposition layers (e.g. 12-1, 12-2, 12-3) forming the inorganic film 12 includes the deposition layers composed of aluminium (III) oxide (Al₂O₃) alternating with the deposition layers composed of at least one metal oxide compound different from aluminium (III) oxide. The at least metal oxide compound different from aluminium (III) oxide can be substituted, in the stack, by a semiconductor oxide (e.g. silica), or, alternatively, by an elemental metal, a nitride compound, a carbide compound, a sulfide compound or any other appropriate chemical compound.

In embodiments, the plurality of deposition layers forming the inorganic film 12 includes the deposition layers composed of aluminium (III) oxide (Al₂O₃) alternating with the deposition layers composed of any one of titanium(IV) oxide (TiO₂), hafnium (IV) oxide (HfO₂), tantalum (V) oxide (Ta₂O₅), and zirconium (IV) oxide (ZrO₂).

By way of example, the coating 10 may comprise a number of essentially porous MLD-deposited polymeric layers 11 alternating with the inorganic ALD film stacks 12 having the following configuration: Al₂O₃+_{X}[(HfO₂—Al₂O₃—ZrO₂—Al₂O₃)]. The MLD layer 11 can be deposited between each ALD deposition layer, i.e. more frequently, or every 2-3 ALD deposition layers (not shown).

Further exemplary stack configurations for the film 12 include, but are not limited to the following (nano)laminates:
Al₂O₃—SiO₂—Al₂O₃—TiO₂;
Al₂O₃—HfO₂—Al₂O₃—ZrO₂;
Al₂O₃—HfO₂—ZrO₂;
TiO₂+[(Al₂O₃—TiO₂)];
Al₂O₃—SiO₂;
Al₂O₃—HfO₂;
Al₂O₃—Ta₂O₅; and
Al₂O₃—Ta₂O₅—Al₂O₃—HfO₂.

In some instances, the above mentioned (nano)laminates may be arranged into repeating stacks, having exemplary composition of x[(12-1)-(12-2)-(12-3)-(12-n)].

To obtain the alumina (Al₂O₃) layers, TMA and water precursors were used. To obtain zirconia (ZrO₂) layers, tetrakis(ethylmethylamino)zirconium (TEMAZr) and water precursors were used. Other ALD precursors reactive with water include, but are not limited to diethyl zinc (DEZ, ZnEt₂); titanium tetrachloride (TiCl4), tetrakis(dimethylamido)titanium (TDMATi), tetrakis(ethylmethylamido)-hafnium (IV) (TEMAHf); metal cyclopentadienyl (Cp) precursors with general formula of Me(RCp)ₓ (e.g. bis(ethylcyclopentadienyl)-magnesium, Mg(EtCp)₂); and tert-butylimido)tris(ethylmethylamido)tantalum (TBTEMTA).

The inorganic films 12 formed using the ALD method are fully conformal to the surface being coated.

It is hereby assumed that a skilled person will be able to reproduce the layered structure 12 composed of other inorganic compounds based on the given examples.

FIG. 2 shows a profiled substrate 20 (wherein S1 relates to plateau regions and S2 relates to three-dimensional profile patterns) coated with a laminate coating embodied at 10′ and implemented as a laminate structure comprising a plurality of deposition layers forming the inorganic film 12 and fabricated using the atomic layer deposition method. Configuration shown on FIG. 2 is realized in an absence of MLD interfacial layers. Exemplary stack configurations for the inorganic film 12 presented hereinabove can be utilized in the coating 10′.

In some configurations, the coating 10′ formed on the substrate 20 may comprise the inorganic film 12 deposited on/over said substrate, said inorganic film forming a plurality of deposition layers arranged into at least one stack, wherein the stack has any one of the following compositions: Al₂O₃—HfO₂; Al₂O₃—Ta₂O₅; Al₂O₃—SiO₂—Al₂O₃—TiO₂, Al₂O₃—HfO₂—Al₂O₃—ZrO₂, Al₂O₃—HfO₂—ZrO₂, TiO₂+[(Al₂O₃—TiO₂)]; and Al₂O₃—Ta₂O₅—Al₂O₃—HfO₂. In described configurations, each deposition layer M1-M4 is composed of an oxide compound; however, other compounds (e.g. elemental metals, nitrides, carbides, sulfides, and the like) may be utilized. Stacks of two, three, four or more deposition layers can be conceived, and these stacks may be repeated atop one another (not shown). Any other (nano)laminate configuration including, but not limited to those described hereinabove can be adopted.

The laminate coating shown on FIG. 2 may further comprise at least one layer 11 composed of essentially porous bulk of material deposited by MLD process (not shown). Said at least one layer composed of essentially porous bulk of material can be deposited directly on the substrate 20, or on top of the inorganic film 12. In configurations involving repeating stacks of inorganic deposition layers (ALD-layers), the MLD layer may be deposited on top of a first stack (shown as M1-M4 on FIG. 2). Alternatively, the MLD layer 11 may be deposited every 2-3 ALD deposition layers (not shown).

In embodiments, the coatings 10, 10′ are fabricated on the substrates 20 configured as medical devices including functional electronic components. In embodiments, the coatings 10, 10′ are fabricated on substrates configured as implantable medical devices, optionally miniature implant devices. In further embodiments, the coatings 10 are fabricated on substrates configured as optionally implantable ultrasonic medical devices, such as a piezoelectric micromachined ultrasonic transducer (PMUT) or a capacitive micromachined ultrasonic transducer (CMUT).

Diagnostic sonography (ultrasonography) is an ultrasound-based diagnostic imaging technique used to visualize subcutaneous body structures including tendons, muscles, joints, vessels and internal organs for possible pathology or lesions. The method is also suitable for imaging soft tissues. Along with non-invasive ultrasonic imaging solutions and/or on-skin applications, also ultrasound powered implantable devices have been developed. The latter utilize wireless power transmission through acoustic waves for remote sensing of physiological environment in soft biological tissues, as an alternative to inductive (near field) and radio-frequency (RF) links.

In above mentioned methods, it is important that a packaging of a transducer or sensor device is ultrasound-compatible, i.e. that the packaging has a minimum effect on acoustic signals. Additionally, especially implantable medical device must be protected with a coating that would be biocompatible and prevent metal ion leakage to the body. By way of example, a PMUT device comprises electrode(s) made of aluminium or molybdenum, and thus requires biocompatible encapsulation.

Biocompatibility and the ion leakage preventing barrier properties are achieved by the coating 10, 10′ comprising a multilayer inorganic (ALD) film stack (12, 12A, 12B). The coating 10, 10′ realized for the ultrasound imaging devices can be realized as a laminate structure comprising a plurality of deposition layers fabricated using the atomic layer deposition method. Such structure can be realized also in an absence of MLD interfacial layers (10′, FIG. 2). Provision of ALD-deposited (nano)laminates 10′ as shown on FIG. 2 with a polymeric cover film 22 may be beneficial in applications requiring highly conformal encapsulation of biocompatible devices, such as for example tissue-compatible soft devices.

ALD deposited film stack (12) configurations particularly suitable for coating the ultrasound medical devices include but are not limited to: Al₂O₃—HfO₂; Al₂O₃—Ta₂O₅; and Al₂O₃—HfO₂—Al₂O₃—Ta₂O₅. Encapsulation of the ultrasound diagnostic devices (e.g. transducers as defined above) with the coatings 10, 10′ comprising the stacks 12 according to these configurations do not cause changes in functionality, e.g. frequency, of the devices. Additionally, it has been observed that these coatings remain intact when the ultrasound device, such as a transducer, is acoustically excited by a pulse with amplitude set to 0.1-10 V (vibration during excitation was about 0.5 nm/V).

In the coating 10, a combination of MLD- and ALD layers form a barrier that prevents harmful environmental agent(s) originating from the local environment surrounding the substrate(s) 20 coated with the coating 10 from penetrating the coating structure at defective and/or damaged sites 30 (FIG. 1B) and from contacting the substrate.

The environmental agents act hereby as species harmful for integrity and/or functionality of the substrate. The nature of these harmful species depends on the environment that surrounds the coated substrate.

In some embodiments, the coating 10 is configured to protect the substrate from destruction caused by water originated from moisture penetrated into the coating at defective and/or damaged sites 30. In present case, environmental water acts as harmful/corrosive species. In some other instances, the harmful environmental species may be ions and/or low-molecular weight compounds, for example, as well as a variety of environmental factors including, but not limited to UV light, heat, oxygen and/or other gases. and the like.

By virtue of combining deposition layers having different physical properties (porosity, texture), as well as chemical compositions, the coating 10 acts as a moisture barrier.

Through selection of layer (chemical) composition and deposition method, the coating 10 is further configured to possess a self-repairing/self-healing functionality.

Reference is made to FIG. 1B showing the coating 10, in which a number of defective and/or localized degradation sites 30 have been formed during deposition and/or during use. The defects include mechanical damages, such as cracks, scratches, etc., as well as localized delamination and/or other damages induced by a variety of factors, such as exposure to moisture and/or ions, UV light, heat, oxygen, and the like.

The defects 30 may originate at a macro-level to deprive the coating of its protective and/or aesthetic properties. Additionally or alternatively, the defects 30 originate at micro- and nano-levels and therefore are not necessarily observable by human eye. Since the inorganic ALD films 12 form a physical barrier, the defects 30 are observed to be mainly formed in the inorganic (ALD) films 12, which can be attributed to their non-porous nature. When the film 12 is ruptured, it loses its conformality, thus enabling various environmental species penetrating into the coating through the damages site 30. In an event of multiple ruptures at different layers of the laminate (see FIG. 1B and damage sites 30 in the films 12A, 12B), the harmful species tend to propagate deeper into the coating until they reach the substrate 20. Local exposure of the substrate to harmful environmental species, e.g. moisture, causes corrosion of the substrate inside the protective coating.

The coating 10 comprises essentially porous (MLD) layers 11 (11A, 11B), which act as so-called decoupling layers in order to separate the conformal inorganic films 12 (12A, 12B) from one another and to obstruct defect propagation through the laminate barrier coating (10).

Hence, in embodiments, the coating 10 is configured to prevent environmental moisture 31, e.g. water molecules, from causing detrimental effects on the underlying substrates when penetrated through the microdefects 30 into the laminate. This is achieved by virtue of constructing the (nano)laminate, in which conditions are established that allow infiltration of essentially free precursors compounds 121, such as unbound, unreacted and/or not fully reacted precursor compounds, into the essentially porous bulk of material during the deposition phase thereof (i.e. during deposition of the MLD layer(s) 11 composed of said essentially porous bulk of material). In the bulk of said porous film 11, the unbound, unreacted and/or partially reacted precursors 121 enter chemical interaction with harmful environmental species 31 penetrated into the coating at defective sites 30 and seal said defective sites through formation of a sealing compound 32 (FIG. 1C).

In exemplary configuration, the coating 10 has been obtained having the structure as elucidated by Table 1.

TABLE 1
Exemplary structure of the coating 10.

| Laminate coating 10 | | | Depth/thickness | |
|---|---|---|---|---|
| ALD, 12B | 12B-3 | Al₂O₃ (M1) | 1-2 | nm |
| | 12B-2 | SiO₂ (M2) | 1-2 | nm |
| | 12B-1 | Al₂O₃ (M1) | 5-20 | nm |
| MLD, 11B | pure organic- or hybrid polymer layer | | 5-20 | nm |
| ALD, 12A | 12A-3 | Al₂O₃ (M1) | 1-2 | nm |
| | 12A-2 | SiO₂ (M2) | 1-2 | nm |
| | 12A-1 | Al₂O₃ (M1) | 5-20 | nm |

| | | | |
|---|---|---|---|
| MLD, 11A | pure organic- or hybrid polymer layer | 5-20 | nm |

| | | |
|---|---|---|
| Substrate | 100-150 | μm |

In configuration shown in Table 1, the Al₂O₃ layers (12A-1, 12A-3, 12B-1, 12B-3) act as a (moisture) barrier, while the SiO₂ layers (12A-2, 12B-2) function as capping layers to prevent hydrolysis of aluminium oxide. The (nano)porous layers 11A, 11B serve as ductile “decoupling” layers capable of retaining the unbound/unreacted precursors infiltrated from the (ALD) films 12.

In the coating structure of a given example, the unbound or not fully reacted precursors 121 (e.g. TMA dimer molecule) infiltrate into the essentially porous layers 11A, 11B in the deposition phase of said essentially porous layers 11A, 11B, where said unbound precursors 121 react with “external” water molecules 31 retained in said layers 11A, 11B before the deposition of inorganic oxide layers 12A, 12B (by “external” water molecules we refer to water molecules penetrated into the coating 10 from the outside of the coated substrate, e.g. with the environmental moisture). Reaction between said unbound precursor 121 and the water molecules, which represent hereby harmful environmental species 31, yields formation of a new sealing compound 32. In the presented example, the reaction between TMA (121) and water (31) yields Al₂O₃ and/or Al(OH)x, acting as the sealing compound 32.

Spots 32 formed with the sealing compound at the defect sites 30 throughout the laminate depth can be viewed as sealing “patches” which block penetration of the harmful environmental species (e.g. moisture-originated water) through the layered coating structure 10 (see FIG. 1C, enlarged box). These “patches” self-assemble within the bulk of the essentially porous MLD layer 11 (11A, 11B) when the substrate coated with the coating 10 is exposed to the environment containing the potentially harmful agents. As mentioned above, the environment can be any environment/ambient that surrounds the coated compounds (substrates coated with the coating 10) intended for storage, transportation and/or operation, which environment contains environmental species (such as diffused water droplets, for example) potentially destructive for integrity/functionality of the coated components.

Said unbound, unreacted or partially (not fully) reacted precursor compounds 121, such as TMA (dimer) molecules, for example, act hereby as “self-healing agents”.

In addition of being capable of self-repairing (self-healing), the coating 10 is also rendered with a desiccant function. The latter is obtained by virtue of chemical interaction between the not fully reacted precursor with liquid/vapour, such as water. Hence, the invention further pertains to use of a laminate coating 10 as a desiccant.

In embodiments, the laminate coating 10 further comprises a primer layer 21 produced by pretreating the substrate 20 prior depositing the essentially porous layer 11 thereon. The pretreatment can be performed by (low-temperature) ALD-deposition of at least one monolayer of alumina (Al₂O₃) using TMA and water precursors optionally associated with a plasma-assisted process. The pretreatment can be further performed by treating the substrate with ozone (O₃) or oxygen (O₂). Any other suitable pretreatment method aiming at enhancing the adhesion of polymer molecules to the substrate can be adopted.

In embodiments, the laminate coating 10, 10′ further comprises a cover film 22 deposited on/over the laminate (10, 10′) as a topmost layer. The topmost cover layer 22 is preferably a polymer film, such as a polydimethylsiloxane (PDMS) film, for example (rf. P1, for FIG. 2). Other examples include, but are not limited to polyurethanes, silicones, and the like.

PDMS is one of the most commonly used polymers for coating medical devices, particularly, implantable devices, due to its high biocompatibility and biostability. One of the major concerns associated with this polymer is its high permeability to water vapour, which leads to water condensation and formation of voids causing corrosion of (metal) substrates. Utilization of PDMS in combination with the laminate coating 10 according to the present disclosure overcomes this drawback.

The polymer cover film 22 can be deposited using dip coating- or (over)molding processes, for example.

For practical reasons, the cover film 22 is typically deposited on/over the ALD-film 12. ALD-films are not porous, and so they act as physical barrier layers; therefore, it is advantageous that the ALD deposition layer 12 is used as an upper layer in the stack 10 (prior to applying the cover film 22 or in an event of implementing the coating 10 without the cover film 22).

The coatings 10, 10′ described above and comprising the polymeric cover film 22 made of e.g. PDMS as a capping layer, possess outstanding electrical insulation properties, which make these coatings particularly suitable for encapsulation of electrically powered medical devices, such as biomedical implants.

Experimental trials conducted by soaking electronic substrates 20 deposited with the laminate coating 10, 10′ comprising the PDMS cover film 22 in PBS (phosphate buffered saline) medium at 87° C. have proved that the substrates remain functional for about 9.5 months. Coatings 10, 10′ comprising the ALD films 12 were deposited at 150° C. on Si/SiO₂ chips with TiN or Al conducting lines (substrate size approximately 3 cm×1 cm). Other substrates included Printed Circuit Boards (PCBs) made of composite materials, such as glass-reinforced epoxy laminate materials of FR4 grade, for example.

By way of example, in accelerated (85° C., PBS) tests with on-line resistance measurements, it has been shown that the substrate samples coated with the (nano)laminate coatings 10, 10′ (deposited at about 200° C.) remained functional until the end of the test (100 days). This duration correlates with a lifespan of the coated substrate in a body environment (37° C.) during an 8-year period.

Mentioned ALD-deposited films comprised a number of deposition layers composed of Al₂O₃ alternating with deposition layers composed of any one or more of HfO₂, ZrO₂, TiO₂, SiO2, Nb₂O₅, Ta₂O₅, and any combination thereof.

In pull tests it was shown that an excellent interface strength exists between the ALD-deposited film 12 and PDMS. On the contrary, adhesion between two polymers (e.g. MLD-deposited polymeric layer 11 and the PDMS layer 22) is markedly weaker.

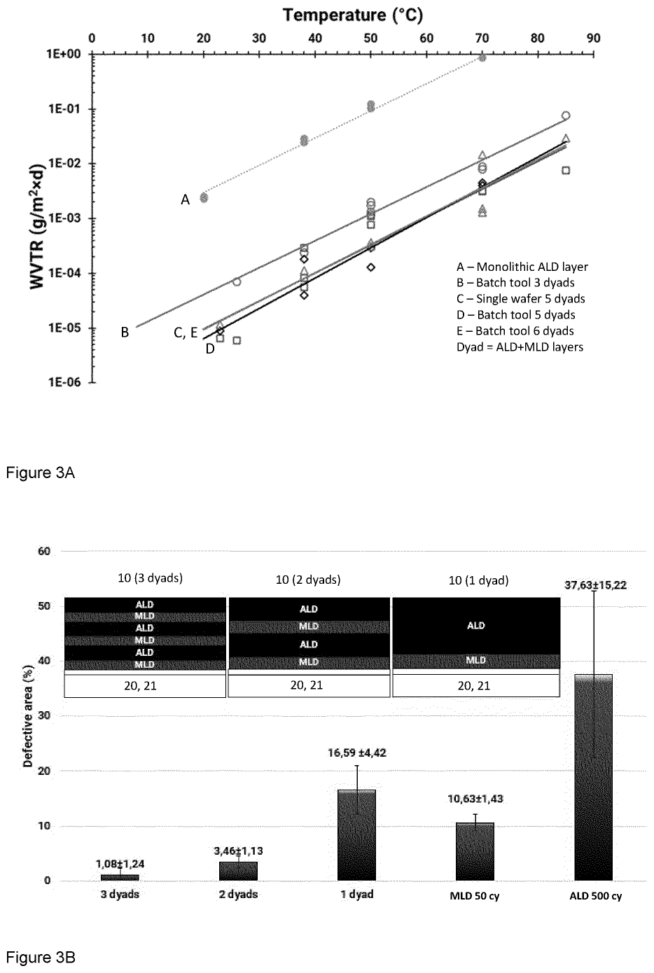

FIG. 3A shows the results for Water vapor transmission rate (WVTR) measurements as a function of temperature (degree Celsius) obtained for the coating 10 deposited with alternating MLD and ALD layers. FIG. 3A shows that ALD/MLD films (B-E) have about two orders of magnitude better WVTR compared to a single (monolithic) ALD film (A) and can reach the WVTR value of: <1E-5 g/m²*d in the ambient. ALD/MLD coatings 10 composed of five or six dyads (C, D, E) show better results than a coating composed of three dyads (B). A dyad is defined, in the present context, with a combination of the essentially porous MLD layer 11 and the inorganic ALD layer 12.

FIG. 3B shows the results of defective area measurements obtained for the laminate coating 10 according to the embodiments, in comparison with monolithic ALD structures. Measurement results of FIG. 3B were obtained for defective areas (%) at 1.96% tensile strain after soaking polycarbonate substrates 20 (PC, 250 μm) coated with the coating 10 (total thickness 50 nm) in acetone at 90° C. The substrate 20 may be provided with a primer layer 21 (interface layer). The coating 10 comprising 2 or 3 stacks (dyads) of MLD-ALD layers (11 and 12) demonstrates markedly reduced defective area coverage in comparison to a non-layered (monolithic) ALD layer (rightmost column). Results shown on FIG. 3B were obtained with the inorganic ALD films composed of Al₂O₃, however, other oxide compounds, such as metal oxides (for example, HfO₂, ZrO₂, TiO₂, Nb₂O₅, Ta₂O₅, or any combination thereof) or silica, demonstrate similar behaviour (also in combination with of Al₂O₃). The essentially porous MLD layer was pure organic- or hybrid polymer layer.

The results shown on FIGS. 3A and 3B confirm that the MLD layers act as “decoupling” layers between the ALD-deposited inorganic barrier films to obstruct defect propagation through the coating and to increase a Water vapor transmission rate (WVTR) lag-time or moisture permeation by creating a tortuous path.

Moreover, when the laminate coating 10 combining the MLD and ALD layers was used instead of a monolithic ALD coating, a lag was observed between detecting cracks in the coating film and detecting damage to the substrate 20 (results not shown). This is indicative of the fact that the substrate remains protected although the individual ALD layers have become damaged/cracked.

Substrate samples coated with the laminate coating 10 combining the MLD and ALD layers are about five times more likely to survive post-deposition cooling and analysis without developing macroscopic defects. Macroscopic defects are detected based on abnormally low activation energy of moisture permeation compared to a baseline. Such samples are analyzed with optical microscope to confirm the presence of macro-defects. Thus, the laminate coating films 10 are more resistant to cracking than (monolithic) ALD films.

The invention further pertains to use of the laminate coating 10 according to the embodiments in packaging, particularly, in medical packaging.

In an aspect, a packaging item, having its surface coated with the coating 10 according to the embodiments is thus provided. The packaging item can be configured as any one of: a container for solids or liquids, optionally a pumped container; a tray, optionally, a multi-compartmental tray; an ampule, a vial, a syringe; a blister pack; an individually wrapped pack, and a pouch.

The laminate coating described herewith may be used on inner and outer surfaces of the medical packaging items. When used on an inner surface of ampules, syringes, etc. the laminate coating may prevent the packaged product from being contaminated by a packaging material (substrate). When used on an outer surface of the packaging items, the laminate coating provides protection from water molecules, protein molecules or clusters, hydroxyl radicals, nitrous oxide species etc., which can originate from the environment surrounding the coated item.

The coating 10, 10′ can be implemented to coat the substrate 20 partially or fully. Full encapsulation is advantageous in protecting medical devices, in particular, implantable medical devices, whereby device failure caused by moisture and/or the corrosive environment in bodily fluids, for example, can be efficiently prevented.

The substrate used in manufacturing of packing items, in particular, medical packing items, and/or medical devices as described herein, may comprise or consist of any one of plastic or glass. Plastic may include polymer materials, including, but are not limited with polyurethane (PU), liquid crystal polymer (LCP), and/or polyethylene terephthalate (PET).

In embodiment, a laminate coating is formed on a substrate, and it comprises: an inorganic film deposited using an atomic layer deposition (ALD) process on/over said substrate, said inorganic film forming a plurality of deposition layers arranged into a stack, the deposition layers composed of aluminium (III) oxide (Al₂O₃) alternating with the deposition layers of hafnium (IV) oxide (HfO₂). In embodiment, a laminate coating formed on a substrate comprises: an inorganic film deposited using an atomic layer deposition (ALD) process on/over said substrate, said inorganic film forming a plurality of deposition layers arranged into a stack, the deposition layers composed of repeating Al₂O₃—SiO2-Al₂O₃—TiO₂ stacks. In embodiment, a laminate coating formed on a substrate comprises: an inorganic film deposited using an atomic layer deposition (ALD) process on/over said substrate, said inorganic film forming a plurality of deposition layers arranged into a stack, the deposition layers composed of repeating Al₂O₃—HfO₂—Al₂O₃—ZrO₂ stacks. In embodiment, a laminate coating formed on a substrate comprises: an inorganic film deposited using an atomic layer deposition (ALD) process on/over said substrate, said inorganic film forming a plurality of deposition layers arranged into a stack, the deposition layers composed of repeating Al₂O₃—HfO₂—ZrO₂ stacks. In embodiment, a laminate coating formed on a substrate comprises: an inorganic film deposited using an atomic layer deposition (ALD) process on/over said substrate, said inorganic film forming a plurality of deposition layers arranged into a stack, the deposition layers composed of repeating TiO₂+[(Al₂O₃—TiO₂)] stacks. In embodiment, a laminate coating formed on a substrate comprises: an inorganic film deposited using an atomic layer deposition (ALD) process on/over said substrate, said inorganic film forming a plurality of deposition layers arranged into a stack, the deposition layers composed of repeating Al₂O₃—Ta₂O₅—Al₂O₃—HfO₂ stacks. In embodiment, a laminate coating formed on a substrate comprises: an inorganic film deposited using an atomic layer deposition (ALD) process on/over said substrate, said inorganic film forming a plurality of deposition layers arranged into a stack, the deposition layers composed of repeating Al₂O₃—Ta₂O₅ stacks.

In embodiments, the laminate coating optionally comprises at least one layer composed of essentially porous bulk of material by an MLD process. In embodiments, the at least one layer composed of essentially porous bulk of material deposited by an MLD process can take place directly onto the substrate, or on top of a first repeating stack.

The invention further pertains to a coated article having its surface coated with the coating 10 according to the embodiments.

The coated article can be configured as a medical device, such as a body implantable medical device, optionally including functional electronic components to develop so-called (bio)electronics solutions. The medical device can thus be configured as an implant absent of electronic components (one-part or multipart implant) or a body implantable medical device that contains electrical/electronic systems, optionally self-powered systems. Implantable medical devices may be designed such as to completely reside in the body, or it may be provided external to the body and connect to an internal organ or another body part. Such devices include, but are not limited to implantable cardiac pacemakers, monitors and defibrillators (e.g. implantable cardioverter defibrillators, ICDs), cochlear implants, a variety of neurological implants and stimulators, infusion pumps, haemodynamic systems, micro-electrical mechanical systems (MEMS), and a variety of (miniaturized) sensors including biosensors for measuring pressure, flow, strain, etc., as well as (bio)chemical sensors, such as the ones for use for example in the treatment of diabetes (e.g. sensors for continuous glucose monitoring, CGM), and other chemically regulated conditions.

In terms of functionality, the implantable devices include a variety of monitoring and/or metering devices, such as the devices for measuring the levels of chemical substances, which serve as markers for certain diseases, in biological fluids, and the devices applicable in treatment of certain conditions or syndromes through neuromodulation (stimulators used in the treatment of migraine, for example) or through regulating the levels of said chemical substances in biological fluids.

The coated article can be also provided as a part or a component used in fabrication of optionally miniaturized sensors and/or semiconductor devices. The item can be further provided as a Printed Circuit Board (PCB) or a PCB assembly (PCBA). The item can be further provided as a non-invasive (medical) device or a part thereof (e.g. a wearable sensor (sensor system) or a semiconductor component/PCB(A)).

Still further, the invention pertains to a medical device, such as a body implantable medical device. The medical devices described hereinabove and similar to those, as well as any related items configured implantable into the body can be conceived. Still further it is appreciated by a person of skill in the art that surface modification is an added advantage of this method such that deposition of one or more ALD layers followed by an MLD layer results in surface functional groups promoting adhesion for a range of subsequent process. As such the method provides applications for catalyst support, solid electrolyte for microbatteries, acts as a template for free-standing films, acts as a water repellent layer on sensors, provides basis for formation of fluorescent thin films, enables gap fill through deposition on a trench structure followed by calcification, acts as a protective barrier film for flexible electronics, acts as a barrier in optics, especially in applications for LEDs, Quantum Dots, and/or nanorod-LEDs, acts as a barrier for nanoparticles, such as phosphor nanoparticles, and acts as a protective barrier film for high-aspect ratio structures and trenches including smart contact lenses, and smart sensors. Additionally or alternatively, the method disclosed hereby can be adapted in manufacturing of flexible smart windows, lithium-ion battery separators (incl. ceramic membrane) and in a variety of space related applications.

It shall be appreciated by those skilled in the art that with the advancement of technology the basic ideas of the present invention may be implemented and combined in various ways. The invention and its embodiments are thus not limited to the examples described hereinabove, instead they may generally vary within the scope of the claims.

1. A method for forming a coating on a substrate, comprising:
(i) using a molecular layer deposition (MLD) process, depositing at least one layer composed of essentially porous bulk of material directly or indirectly on a surface of the substrate, and
(ii) using an atomic layer deposition (ALD) process, depositing an inorganic film on/over the at least one layer formed in step (i),
whereby a coating is formed, wherein, in said at least one layer composed of essentially porous bulk of material, unbound, unreacted and/or partially reacted precursors enter chemical interaction with harmful environmental species penetrated into the coating at defective sites thereof and seal said defective sites through formation of a sealing compound.

2. The method of claim 1, wherein the inorganic film formed in step (ii) comprises at least one deposition layer.

3. The method of claim 1, wherein the inorganic film formed in step (ii) comprises a plurality of deposition layers arranged into a stack, wherein each deposition layer in the stack has the same or different composition.

4. The method of claim 1, wherein the deposition layer or layers forming the inorganic film in step (ii) are composed of any compound selected from the group consisting of: aluminium (III) oxide (Al₂O₃), titanium(IV) oxide (TiO₂), hafnium (IV) oxide (HfO₂), tantalum (V) oxide (Ta₂O₅), zirconium (IV) oxide (ZrO₂), and silicon dioxide (SiO₂).

5. The method of claim 3, wherein the plurality of deposition layers forming the inorganic film in step (ii) includes the deposition layers composed of aluminium (III) oxide (Al₂O₃) alternating with the deposition layers composed of a metal oxide compound different from aluminium (III) oxide.

6. The method of claim 5, wherein the plurality of deposition layers forming the inorganic film in step (ii) includes the deposition layers composed of aluminium (III) oxide (Al₂O₃) alternating with the deposition layers composed of any one of titanium(IV) oxide (TiO₂), hafnium (IV) oxide (HfO₂), tantalum (V) oxide (Ta₂O₅), and zirconium (IV) oxide (ZrO₂).

7. The method of claim 3, wherein the plurality of deposition layers forming the inorganic film in step (ii) includes the deposition layers composed of aluminium (III) oxide (Al₂O₃) alternating with the deposition layers of hafnium (IV) oxide (HfO₂).

8. The method of claim 1, comprising repeating steps (i) and (ii) until the coating having a desired total thickness has been formed.

9. The method of claim 1, wherein the step (ii) is performed before the step (i).

10. The method of claim 1, further comprising pretreatment of the substrate.

11. The method of claim 10, wherein pretreatment of the substrate comprises treating the substrate with any one of: ozone (O₃), oxygen (O₂), and/or by depositing onto the substrate surface a primer layer that enhances adhesion of the essentially porous material layer formed in step (i) to the surface of the substrate.

12. The method of claim 1, further comprising depositing a polymer film on/over the coating as a topmost layer.

13. The method of claim 12, wherein the polymer film constituting the topmost layer consists of polydimethylsiloxane (PDMS) or polyurethane (PU).

14. The method of claim 1, wherein the harmful environmental species comprise any one of: water molecules, hydroxyl radicals, nitrous oxide species, biological molecules, and the like, originated from an environment surrounding the substrate(s) coated with the laminate coating and penetrated into the coating at the defective sites.

15. A self-healing laminate coating formed on a substrate and comprising:
(a) at least one layer composed of essentially porous bulk of material deposited using a molecular layer deposition (MLD) process, and
(b) an inorganic film deposited using an atomic layer deposition (ALD) process,
wherein, in said at least one layer composed of essentially porous bulk of material, unbound, unreacted and/or partially reacted precursors enter chemical interaction with harmful environmental species penetrated into the coating at defective sites thereof and seal said defective sites through formation of a sealing compound.

16. The laminate coating of claim 15, wherein the inorganic film (b) comprises at least one deposition layer.

17. The laminate coating of claim 15, wherein the inorganic film (b) comprises a plurality of deposition layers arranged into a stack, wherein each deposition layer in the stack has same or different composition.

18. The laminate coating of claim 15, wherein the deposition layer or layers forming the inorganic film (b) are composed of any compound selected from the group consisting of: aluminium (III) oxide (Al₂O₃), titanium(IV) oxide (TiO₂), hafnium (IV) oxide (HfO₂), tantalum (V) oxide (Ta₂O₅), zirconium (IV) oxide (ZrO₂), and silicon dioxide (SiO₂).

19. The laminate coating of claim 15, wherein the inorganic film (b) comprises deposition layers composed of aluminium (III) oxide (Al₂O₃) alternating with the deposition layers composed of a metal oxide compound different from aluminium (III) oxide.

20. The laminate coating of claim 15, wherein the inorganic film (b) comprises deposition layers composed of aluminium (III) oxide (Al₂O₃) alternating with the deposition layers composed of any one of titanium(IV) oxide (TiO₂), hafnium (IV) oxide (HfO₂), tantalum (V) oxide (Ta₂O₅), and zirconium (IV) oxide (ZrO₂).

21. The laminate coating of claim 15, wherein the inorganic film (b) comprises deposition layers composed of aluminium (III) oxide (Al₂O₃) alternating with the deposition layers of hafnium (IV) oxide (HfO₂).

22. The laminate coating of claim 15, wherein the inorganic film (b) comprises deposition layers composed of aluminium (III) oxide (Al₂O₃) alternating with the deposition layers of tantalum (V) oxide (Ta₂O₅).

23. The laminate coating of claim 1, wherein the inorganic film (b) is deposited on/over the substrate and/or on/over the at least one layer of essentially porous material.

24. The laminate coating of claim 15, comprising the inorganic film (b) deposited using the atomic layer deposition (ALD) process on/over the substrate, wherein the inorganic film (b) is formed with a plurality of deposition layers arranged into at least one stack having a composition of: Al₂O₃—SiO2-Al₂O₃—TiO₂.

25. The laminate coating of claim 15, comprising the inorganic film (b) deposited using the atomic layer deposition (ALD) process on/over the substrate, wherein the inorganic film (b) is formed with a plurality of deposition layers arranged into at least one stack having a composition of: Al₂O₃—HfO₂—Al₂O₃—ZrO₂.

26. The laminate coating of claim 15, comprising the inorganic film (b) deposited using the atomic layer deposition (ALD) process on/over the substrate, wherein the inorganic film (b) is formed with a plurality of deposition layers arranged into at least one stack having a composition of: Al₂O₃—HfO₂—ZrO₂.

27. The laminate coating of claim 15, comprising the inorganic film (b) deposited using the atomic layer deposition (ALD) process on/over the substrate, wherein the inorganic film (b) is formed with a plurality of deposition layers arranged into at least one stack having a composition of: TiO₂+[(Al₂O₃—TiO₂)].

28. The laminate coating of claim 15, comprising the inorganic film (b) deposited using the atomic layer deposition (ALD) process on/over the substrate, wherein the inorganic film (b) is formed with a plurality of deposition layers arranged into at least one stack having a composition of: Al₂O₃—Ta₂O₅—Al₂O₃—HfO₂.

29. The laminate coating of claim 15, comprising the inorganic film (b), in which the deposition layers are arranged into repeating stacks.

30. The laminate coating of claim 24, further comprising at least one layer composed of essentially porous bulk of material deposited using a molecular layer deposition (MLD) process.

31. The laminate coating of claim 15, wherein the at least one layer composed of essentially porous bulk of material is deposited directly on the substrate, or on top of the inorganic film (b).

32. The laminate coating of claim 15, further comprising a primer layer formed on the substrate surface to enhance adhesion of the essentially porous material layer (a) to the surface of the substrate.

33. The laminate coating of claim 15, further comprising a polymer film deposited on/over the coating as a topmost layer.

34. The laminate coating of claim 33, wherein the polymer film constituting the topmost layer consists of polydimethylsiloxane (PDMS).

35. The laminate coating of claim 15, wherein the substrate is selected from the group consisting of: a medical device, a medical packaging, an Organic Light Emitting Diode (OLED), and a sensor.

36. The laminate coating of claim 15, wherein the sealing compound formed in the essentially porous bulk of material is a product of chemical interaction between the unbound, unreacted and/or partially reacted precursors and harmful environmental species, wherein said harmful environmental species comprise any one of: water molecules, hydroxyl radicals, nitrous oxide species, biological molecules, and the like, originated from an environment surrounding the substrate(s) coated with the laminate coating and penetrated into the coating at the defective sites.

37. A desiccant comprising the laminate coating as defined in claim 15.

38. A packaging item comprising the laminate coating as defined in claim 15.

39. A catalyst support, solid electrolyte for microbatteries, lithium-ion battery separators, free-standing films, water repellent layer on sensors, fluorescent thin films, barrier film for flexible electronics, barrier film for optics, barrier film for nanoparticles/phosphors, flexible smart windows, smart contact lenses, smart sensors, or space related applications, comprising the laminate of claim 15.

40. A packaging item, having its surface coated with a laminate coating according to claim 15.

41. The packaging item of claim 40 selected from the group consisting of: a container, a tray, an ampule, a vial, a syringe, a blister pack, an individually wrapped pack, and a pouch.

42. The packaging item of claim 40, comprising glass or polymer, liquid crystal polymer (LCP), and/or polyethylene terephthalate (PET).

43. A medical device having its surface coated with a laminate coating according to claim 15.

44. The medical device of claim 43 configured as an ultrasonic medical device.
